(19) United States
(12) Patent Application Publication
(10) Pub. No.: US 2006/0089526 A1
(43) Pub. Date: Apr. 27, 2006

(54) Self-sealing closure for an ophthalmic cannula

(75) Inventors: David E. Chen, Fremont, CA (US); Erik W. Peterson, Walnut Creek, CA (US); Steven W. Smith, Alameda, CA (US)

Correspondence Address: MICHAEL BEST & FRIEDRICH, LLP, 100 E WISCONSIN AVENUE, MILWAUKEE, WI, 53202, US

(73) Assignee: Medical Instrument Development Laboratories, Inc., San Leandro, CA (US)

(21) Appl. No.: 10/970,792

(22) Filed: Oct. 21, 2004

(51) Int. Cl. A61B 1/00

(52) U.S. Cl. 600/101; 606/108; 277/628; 604/167.01

(57) ABSTRACT

A self-sealing closure device adapted to selectively seal an extra-ocular end of a cannula. The closure is designed to prevent the flow of fluids through the cannula in the absence of an instrument being positioned within the cannula. The elastic closure includes a first portion that extends at least partially around an outside surface of the extra-ocular end of the cannula and a second portion that extends at least partially within the extra-ocular end of the cannula. A passageway extends through the second portion of the closure and is selectively sealed by a septum having multiple elastic flaps. The flaps are selectively moveable to control the movement of fluids through the elastic closure.

### FIELD OF THE INVENTION

Embodiments of the invention relate to aspects of improved trocar-cannula devices for use in surgery of the posterior segment of the eye.

### BACKGROUND OF THE INVENTION

A trocar-cannula is a surgical instrument. It can be used to drain fluid from a body cavity, introduce fluids into the body cavity, and insert a tool into the body cavity to perform surgical functions. The trocar-cannula is comprised of two principal parts: (1) a hollow tube or cannula and (2) a puncturing member referred to as an obturator or trocar. The cannula is inserted through the wall of the body cavity with the assistance of the trocar as it is passed through the cannula.

A trocar-cannula may be used in cardiovascular surgery, laparoscopic surgery, arthroscopic surgery, and intraocular surgery. However, the construction of the trocar-cannula is generally different for each application. For example, an arthroscopic trocar-cannula is usually too large for intraocular surgery. Furthermore, it may contain projections on the exterior surface of the lumen to prevent the cannula from inadvertently disengaging the joint. These projections could cause tissue damage if applied to the eye or other parts of the body.

In intraocular surgery, a trocar-cannula is often used to obtain access to the posterior-segment of the eye (area behind the lens). During posterior-segment surgery, one or more trocar-cannulas can be used. Multiple trocar-cannulas are used during some surgeries to create several, small penetrations of the sclera rather than one large penetration. When several trocar-cannulas are used, one cannula can be used as an infusion cannula to infuse fluids into the cavity, while the other cannulas can be used for surgical instruments, such as a suction-cutter, forceps, or a light. Although surgical instruments can be introduced directly through incisions in the sclera, some surgeons introduce instruments through a cannula to reduce damage to the incision and nearby tissues.

### SUMMARY OF THE INVENTION

One aspect of the invention is directed toward a self-sealing ophthalmic cannula. During intraocular surgery, fluids within the eye can leak out of the eye via the cannula unless the cannula is plugged with an instrument or plug. Many conventional ophthalmic cannulas are not self-sealing. Rather, a surgeon must manipulate plugs to selectively seal the cannula to prevent leakage from the eye through the cannula. The manipulation of these plugs can be time consuming. Furthermore, since the plugs can be separated from the cannula, the plugs can be lost during surgery.

In one embodiment, the ophthalmic cannula has a closure device that is coupled to the extra-ocular end of the cannula. The closure device is designed to prevent the flow of fluids through the cannula in the absence of an instrument being positioned within the cannula.

The elastic closure includes a first portion that extends at least partially around an outside surface of the cannula and a second portion that extends at least partially within the extra-ocular end of the cannula. A passageway extends through the second portion of the closure and is selectively sealed by a septum having an opening defined by one or more slits. The slits at least partially define elastic flaps that are selectively moveable to control the movement of fluids through the elastic closure. An obturator or other surgical instrument can pass through the passageway in the elastic closure by displacing the elastic flaps upon insertion. When the obturator or other instrument is withdrawn, the flaps close to seal the passageway and prevent fluids from flowing through the passageway.

The passageway and septum in the closure can be sized such that the instrument can slide in and out with minimal friction. The passageway or septum can also form a seal against the instrument to prevent fluids from leaking between the instrument and the closure.

In some embodiments, a connector for infusion tubing or optical fiber can be inserted into the opening of the elastic closure and securely held by the force of the stretched elastic material. The connector can be larger in diameter than the instruments that pass through the cannula to further stretch the elastic material.

These and other aspects of the embodiments of the invention, together with the organization and operation thereof, will become apparent from the following detailed description when taken in conjunction with the accompanying drawings.

### BRIEF DESCRIPTION OF THE DRAWINGS

FIG. 1 is a cross-sectional view of an exemplary cannula assembly embodying aspects of the invention.

FIG. 2 is a cross-sectional view of the self-sealing cannula assembly of FIG. 1 with an obturator positioned in the lumen of the cannula assembly.

FIG. 3 is a cross-sectional view of the cannula assembly of FIG. 1 with a connector for infusion tubing positioned in the closure.

FIG. 4 is a cross-sectional view of the cannula assembly of FIG. 1 with a connector for optical fiber positioned in the closure.

FIGS. 5A and 6A are perspective views of the penetrating end of the obturator according to two alternative embodiments.

FIGS. 5B and 6B are plan views of the aperture through the elastic septum caused by the penetrating end of the corresponding obturator shown in FIGS. 5A and 6A respectively.

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limited. The use of “including,” “comprising” or “having” and variations thereof is meant to encompass the items listed thereafter and equivalents thereof as well as additional items. The terms “mounted,” “connected,” and “coupled” are used broadly and encompass both direct and indirect mounting, connecting and coupling. Further, “connected” and “coupled” are not restricted to physical or mechanical connections or couplings.

### DETAILED DESCRIPTION

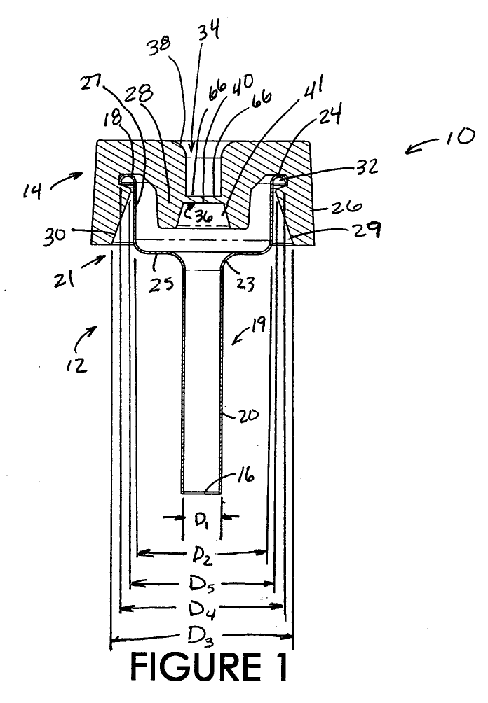

With reference to FIG. 1, there is shown a cannula assembly or device 10 of an ophthalmic trocar-cannula. The cannula assembly 10 includes a sleeve or cannula 12 and a self-sealing closure 14. In normal surgical use, a first end 16 of sleeve 12 communicates with the interior of the eye, while a second end 18 is positioned exterior to the eye (an extra-ocular end). The closure 14 is coupled to the second end 18 of sleeve 12.

In the illustrated embodiment, the sleeve 12 has a first portion 19 extending from the first end 16 and a second portion 21 in communication with the first portion 19. The first portion 19 at least partially includes an elongated tube 20 extending in an axial direction. The elongated tube 20 has a first diameter D₁. The second portion 21 at least partially includes a receptacle or chamber 22 having a second diameter D₂ that is greater than the first diameter. A lip 24 is positioned at the second end 18 of the sleeve 12 adjacent the receptacle 22. The elongated tube 20 is axially aligned with and coupled to the receptacle 22 for fluid communication. The receptacle 22 joins the elongated tube 20 at a radius 23 that forms a transition between the different diameters D₁, D₂ of the receptacle 22 and the elongated tube 20.

The receptacle 22 has a base 25 and at least one wall 27 coupled to the perimeter of the base 25. The base 25 extends from the tube 20 in a substantially radial direction. In some embodiments, the base 25 is circular. A single wall 27 is coupled to the outer perimeter of the base 25 and extends around the perimeter of the base 25. However, in other embodiments, the base 25 can be configured differently and consequently may require more than one wall 27 to define the receptacle 22. The wall 27 extends in a substantially axial direction, and the lip 24 is coupled to the wall 27 at the second end 18 of the sleeve 12.

The sleeve 12 can be fabricated from several different types of materials and can be fabricated several different ways. For example, some embodiments can be formed as a single deep-drawn metal part, such as the illustrated embodiment. However, in other embodiments the sleeve 12 can be fabricated from separate parts employing various materials.

The closure 14 is composed of an elastomer or similar elastic material. The closure 14 is adapted to engage and selectively self-seal the second end 18 of the sleeve 12. The closure 14 comprises a first portion or an attachment ring 26 and a second portion or a central boss 28 coupled to the attachment ring 26. As illustrated, the attachment ring 26 and the central boss 28 have a unitary construction. The attachment ring 26 and the central boss 28 at least partially define a recess 29 adapted to receive the second end 18 of sleeve 12. The attachment ring 26 is adapted to extend over at least a portion of the outside surface of the receptacle 22. The central boss 28 is adapted to extend at least partially within the receptacle 22.

The attachment ring 26 includes a tapered lead-in 30 partially defining the recess 29. The tapered lead-in 30 is adapted to engage the lip 24 of the sleeve 12 as the closure 14 is applied to the sleeve 12. As illustrated, the tapered lead-in 30 extends from a bottom surface of the closure 14 in both the radial and axial direction to form the taper. The tapered lead-in 30 of the illustrated embodiment has a diameter D₃ adjacent the bottom surface of the closure 14 that is slightly larger than the diameter D₄ of the lip 24. This helps to initiate the application of the closure 14 to the sleeve 12. The other end of the tapered lead-in 30 has a diameter D₅ that is less the diameter D₃ adjacent the bottom surface of the closure 14 and about the same as the diameter D₂ of the receptacle 22. An undercut 32 is positioned adjacent the smaller diameter D₅ portion of the tapered lead-in 30 and has a diameter that is about the same as the lip 24. The combination of the small diameter D₅ portion of the tapered lead-in 30 and the relatively larger diameter undercut is adapted to retain the closure on the sleeve 12 by engaging the lip 24.

The cannula assembly 10 is assembled by pressing the second end 18 of the sleeve 12 into the recess 29 of the closure 14. The lip 24 engages the tapered lead-in 30 and slides along the lead-in 30 to cause the attachment ring 26 to elastically expand and deform in the radial direction. Eventually, the lip 24 slides past the tapered lead-in 30 and engages in undercut 32. As the lip 24 disengages the tapered lead-in 30 and enters the undercut 32, the attachment ring 26 returns substantially to its previous shape to secure the undercut 32 to the lip 24.

A passageway 34 is positioned within the central boss 28 of the closure 14. Specifically, the passageway 34 is disposed along the axis of central boss 28 to define a flow path through the closure 14. The passageway 34 is substantially aligned with tube 20 of the sleeve 12 and has substantially the same diameter as the tube 20. The passageway 34 includes a tapered entry 38, which can aid in the insertion of an instrument into the passageway 34. In the embodiments shown, the passageway 34 is elastic, which also helps in the insertion and retention of instruments.

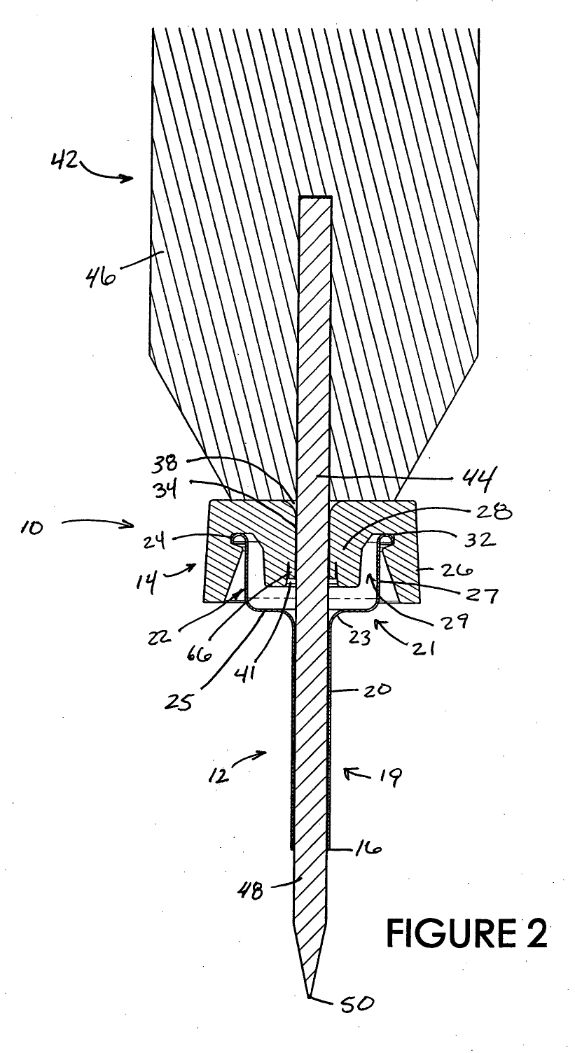

A septum 36 is positioned within the passageway 34, below the tapered entry, to selectively seal the passageway 34 and prevent fluid flow through the passageway 34. The septum 36 has an aperture 40 that is defined by a plurality of elastic flaps 66 that meet at one or more slits. In some embodiments, the flaps 66 are connected to the wall of the passageway 34 in a cantilevered manner and have a portion that is selectively movable relative to the wall. In the embodiment shown, the flaps extend substantially perpendicularly to the vertical walls of the passageway 34. The portions of the flaps 66 move relative to the wall in a pivoting or bending manner between a closed position (shown in FIG. 1) and a plurality of open positions (shown in FIGS. 2-4). In the closed position, the flaps 66 rest in an elastically biased position to substantially obstruct the flow of fluids through the passageway 34. In the open position, portions of the flaps 66 are pivoted or bent relative to the closed position to allow fluids or other items to pass through the passageway 34. The cantilevered portions of the flaps 66 move from the closed position to the open position in response to contact from an instrument inserted into the passageway 34. Upon removal of the instrument, the flaps 66 return to the closed position due to elastic forces and prevent the flow of fluid through the sleeve 12.

As illustrated in FIGS. 1 and 2, the passageway 34 also has a tapered or recessed portion 41 positioned adjacent the septum 36. The recessed portion 41 is positioned downstream of the septum 36 relative to the insertion direction of an instrument into the passageway 34. The recessed portion 41 is dimensioned to receive the flaps 66 of the septum 36 as they are moved from the rest position in the passageway 34 by the insertion of a tool into the passageway 34. As illustrated in FIG. 2, recessed portion 41 allows the flaps 66 to be pivoted or bent to a position were the flaps 66 substantially do not obstruct any portion of the passageway 34. In other words, the flaps 66 are allowed to move to a position were the flaps partially define an opening having substantially the same diameter as the non-tapered portion of the passageway 34. In the pivoted or bent position, the flaps 66 remain biased against the instrument to at least partially form a seal around the instrument.

The cannula assembly 10 is inserted into an eye with an obturator 42. With reference to FIG. 2, the cannula assembly 10 is shown assembled to the obturator 42, as it would be for insertion into the eye. The obturator 42 comprises a needle 44 and a handle 46 coupled to the needle 44. The needle 44 has a cylindrical shaft 48 and a pointed tip 50. The needle 44 is passed through the passageway 34 in the closure 14 and through the tube 20 of the sleeve 12, both of which are slightly larger in diameter than the needle 44. Entry of the needle 44 causes the aperture 40 in the septum 36 to be enlarged and also causes the septum 36 to be elastically deformed and displaced to the side of the needle 44, as shown in FIG. 2.

During insertion of the cannula assembly 10 into an eye, the pointed tip 50 of the needle 44 punctures the surface of the eye. The handle 46 of obturator 42 is then pressed forward to further drive the needle 44 into the eye. As the needle 44 is further inserted into the eye, the tube 20 of the cannula 12 is inserted into the puncture until the receptacle 22 of the cannula 12 rests against the surface of the eye. The obturator 42 can then be withdrawn from the eye and the cannula assembly 10 to leave the cannula assembly 10 retained in the eye. The force of the elastically deformed tissues surrounding the puncture helps to retain the cannula assembly 10 in the eye.

When the needle 44 of the obturator 42 is withdrawn from the closure 14, the septum 36 elastically returns to its original at rest position, as shown in FIG. 1, to thereby restore the aperture 40 to its closed position. In the closed position, the passageway 34 is substantially sealed to prevent the flow of fluid through the cannula assembly 10.

The aperture 40 through the septum 36 can be formed by a variety of methods. For example, it can be formed during the molding operation, at other times prior to assembly of the cannula assembly 10, or after assembly of the cannula assembly 10. In some embodiments, the closure 14 is molded with an uninterrupted septum 36 and the aperture 40 is formed in a secondary operation. Specifically, in some embodiments, the aperture 40 is formed by puncturing the septum 36 with the pointed tip 50 of needle 44 as the cannula assembly 10 is assembled onto the obturator 42.

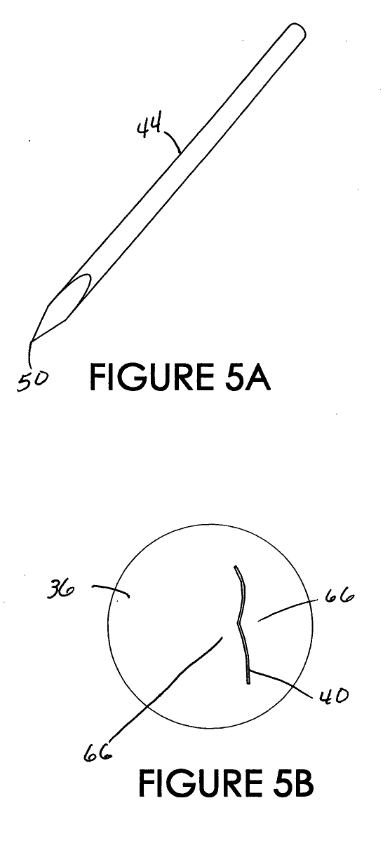

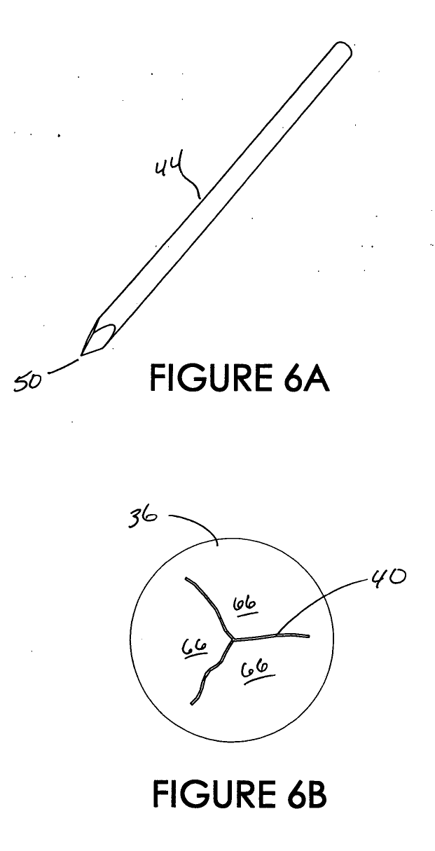

The shape and sealing characteristics of the aperture 40 can be dependent upon the type of needle 44 used to from the aperture. With reference to FIGS. 5 and 6, two different pointed tips 50 of the needle 44 are illustrated. FIG. 5A shows a tip 50 formed by grinding one angled face entirely across the needle 44 and then sharpening the point with two smaller faces, resulting in an off-center tip and two sharp edges. FIG. 5B shows the aperture 40 resulting from the passage of this needle configuration through the septum 36. As illustrated, the aperture 40 is a single, somewhat curviform slit. FIG. 6A shows a tip 50 formed by grinding three angled faces onto the needle 44, resulting in an on-center tip and three sharp edges. FIG. 6B shows the aperture 40 resulting from the passage of this needle configuration through the septum 36. As illustrated, the aperture 40 includes three intersecting slits that define three flaps 66. The seal formed in FIG. 5 can be slightly more effective than the seal illustrated in FIG. 6 at blocking fluid flow through septum 36. However, the seal illustrated in FIG. 5 also requires slightly greater force to insert surgical instruments. Other configurations of the aperture 40 can also be used.

Other surgical instruments similar in size and shape to the shaft 48 of needle 44 can also be passed through the cannula assembly 10. Also, surgical instruments such as cutters, forceps, lights, and the like can be inserted through the passageway. When such instruments are withdrawn from the cannula assembly 10, the egress of fluids from the eye is substantially blocked by the septum 36 elastically returning to its rest position to substantially seal the passageway 34.

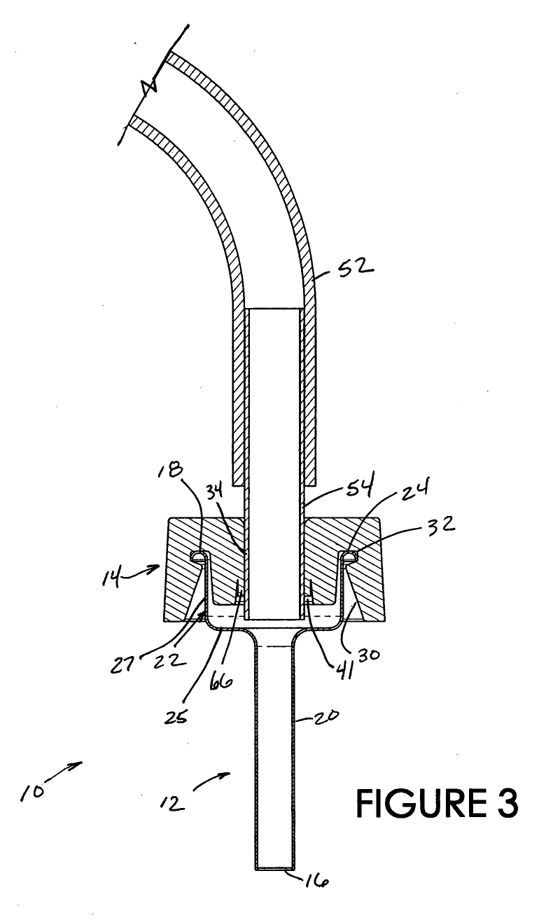

FIG. 3 illustrates an exemplary embodiment of the cannula assembly 10 being used as an infusion cannula. The infusion cannula includes the cannula assembly 10, a connector 54 coupled to the cannula assembly 10, and flexible tubing 52 coupled to the connector 54. The flexible tubing 52 is connected to a source of infusion fluid, not shown in FIG. 3.

After the cannula assembly 10 has been placed in the eye, the connector 54 is inserted into the tapered entry 38 of the closure 14 and then pressed further into the passageway 34. The diameter of the connector 54 is greater than the diameter of passageway 34. Accordingly, the central boss 28 of closure 14 is elastically deformed. The resulting elastic forces cause the connector 54 to be retained in the closure 14 of cannula assembly 10. In the illustrated embodiment, the connector 54 is a simple tube. The connector 54 of some embodiments can include ridges or other features to better retain it in the cannula assembly 10.

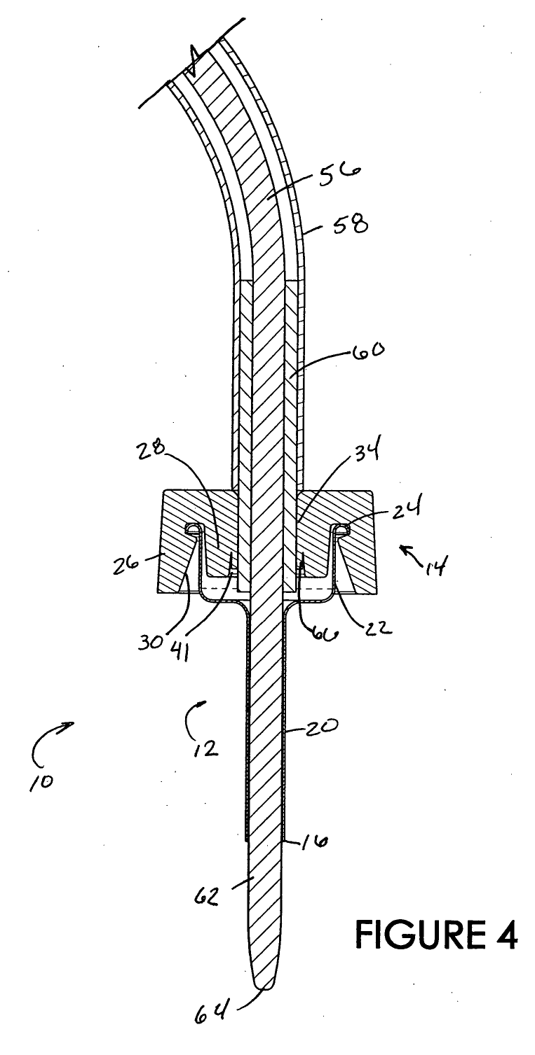

FIG. 4 illustrates an exemplary embodiment of the cannula assembly 10 being used with a fixed fiberoptic illuminator. The fiberoptic illuminator includes an optical fiber 56, protective sheath 58, and connector 60. The connector 60 is coupled to the passageway 34 and the protective sheath 58 is coupled to connector 60. The optical fiber 56 is positioned inside the protective sheath 58 and the connector 60. The optical fiber 56 has an extension 62 that extends through the connector 60 and terminates at a tip 64, which is designed to diffuse conducted light. The optical fiber 56 is also coupled to a source of light, not shown in FIG. 4.

After the cannula assembly 10 has been placed in the eye, the extension 62 of optical fiber 56 is inserted through the passageway 34 in the closure 14 and through the tube 20 of the sleeve 12. The connector 60 is then pressed into the passageway 34. The diameter of connector 60 is greater than the diameter of the passageway 34. Accordingly, the central boss 28 of the closure 14 is elastically deformed. The resulting elastic forces cause the connector 60 to be retained in cannula assembly 10. The illustrated connector 60 is a simple tube. However, in other embodiments, the connector 60 could also include ridges or other features to better retain it in the cannula assembly 10.

The embodiments described above and illustrated in the figures are presented by way of example only and are not intended as a limitation upon the concepts and principles of the present invention. As such, it will be appreciated by one having ordinary skill in the art that various changes in the elements and their configuration and arrangement are possible without departing from the spirit and scope of the present invention. For example, in some instances, the cannula assembly has been described as having a circular tube configuration with respect to one or more potions of the cannula 12 or closure 14. However, in some embodiments, one or more portions can have a non-circular tube configuration, such as the receptacle 22 and portions of the closure 14. Accordingly, references to diameters should be understood as also including other similar measurements for non-circular configurations. In addition, the closure 14 may be secured to the sleeve or cannula 12 in multiple ways. For example, surgical adhesive or other suitable adhesives may be used to secure the closure 12 to the cannula 12.

In an alternative embodiment, the lip 32 of the cannula 12 may be removed or reshaped. In addition, the tapered lead-in 30 may be reshaped such that one or more abutting-surface locations are formed. For example, if the tapered lead-in 30 is formed to be substantially vertically-oriented, the wall 27 and lead-in 30 may be configured such that they abut oneanother around a circumference. The friction fit between the two or added adhesive may be sufficient to secure the closure 14 to the cannula 12.

Various features of the invention are set forth in the following claims.

1. An ophthalmic surgical device comprising:
a tube extending in an axial direction;
a receptacle coupled to the tube, the receptacle including:
a base coupled to the elongated tube and extending substantially radially away from the tube;
a wall coupled to the base and extending away from the base substantially in the axial direction; and
a lip coupled to the wall and extending at least partially in a radial direction;
an elastic closure configured to be coupled to the receptacle, the elastic closure including:
a tapered surface partially defining a recess, the tapered surface extending partially in the axial direction and partially in the radial direction, the tapered surface adapted to engage the lip of the receptacle;
an undercut positioned adjacent the tapered surface and partially defining the recess, the undercut extending further in the radial direction than an immediately adjacent portion of the tapered surface, the undercut being positioned and dimensioned to receive the lip of the receptacle;
a boss partially defining the recess, the boss dimensioned to be at least partially received within the receptacle and having an elongated passageway substantially axially aligned with the tube; and
a septum positioned within the elongated passageway of the boss, portions of the septum configured to move within the passageway to selectively seal the passageway.

2. The device of claim 1, wherein the elongated passageway has a tapered portion positioned adjacent the septum and adapted to selectively receive portions of the septum.

3. The device of claim 2, wherein the tapered portion is dimensioned to allow the septum to be substantially entirely moved from a position extending into a non-tapered diameter of the passageway.

4. The device of claim 1, wherein the septum comprises a plurality of elastic flaps.

5. The device of claim 4, wherein the flaps are defined by intersecting slits extending through the septum.

6. The device of claim 1, wherein the passageway is disposed coaxially with the tube.

7. The device of claim 1, wherein the wall extends around a perimeter of the base and forms a cylinder.

8. The device of claim 1, wherein the tapered surface extends over an outside surface of the receptacle.

9. The device of claim 8, wherein the tapered surface extends further from the lip than the boss in the axial direction.

10. The device of claim 1, wherein the tapered surface, the boss, and the septum are a unitary structure.

11. A self-sealing closure device for an ophthalmic cannula, where the ophthalmic cannula has a first end and a second end having a lip, a first portion of the ophthalmic cannula extends from the first end, a second portion of the ophthalmic cannula extends from the second end and has a chamber in fluid communication with the first portion, the self-sealing closure device comprising:
an elastic body;
a tapered surface partially defining a recess in the body, the tapered surface extending partially in an axial direction and partially in a radial direction, the tapered surface adapted to engage the lip of the second end of the cannula;
an undercut positioned adjacent the tapered surface and partially defining the recess, the undercut extending further in the radial direction than an immediately adjacent portion of the tapered surface, the undercut being positioned and dimensioned to receive the lip of the cannula;
a boss partially defining the recess, the boss dimensioned to be at least partially received within the chamber of the second portion and having an elongated passageway; and
a septum positioned within the elongated passageway of the boss, portions of the septum configured to move within the passageway to selectively seal the passageway.

12. The self-sealing closure device of claim 11, wherein the elongated passageway has a tapered portion positioned adjacent the septum and adapted to selectively receive portions of the septum.

13. The self-sealing closure device of claim 12, wherein the tapered portion is dimensioned to allow the septum to be substantially entirely moved from a position extending into a non-tapered diameter of the passageway.

14. The self-sealing closure device of claim 11, wherein the septum comprises a plurality of elastic flaps.

15. The self-sealing closure device of claim 14, wherein the flaps are defined by intersecting slits extending through the septum.

16. The self-sealing closure device of claim 11, wherein the tapered surface is adapted to extend over an outside surface of the chamber.

17. The self-sealing closure device of claim 16, wherein the tapered surface is dimensioned to extend further from the lip than the boss in the axial direction.

18. The self-sealing closure device of claim 11, wherein the tapered surface, the boss, and the septum are a unitary structure.

19. A self-sealing closure device for an ophthalmic cannula, where the ophthalmic cannula has a first end and a second end, a tube that extends from the first end and a chamber that extends from the second end and is in fluid communication with the tube, the self-sealing closure device comprising:
an elastic body having a first portion and a second portion at least partially defining a recess, the first portion having a surface adapted to extend along a portion of an outside surface of the chamber, the second portion adapted to extend into the chamber, the first portion extending further in an axial direction than the second portion;
an elongated passageway adapted to substantially align with the tube of the cannula; and
a septum positioned within the elongated passageway, portions of the septum configured to move within the passageway to selectively seal the passageway.

20. The self-sealing closure device of claim 19, wherein the surface is a tapered surface coupled to the first portion of the unitary body and at lease partially defining the recess, the tapered surface extending partially in the axial direction and partially in a radial direction, the tapered surface adapted to engage the second end of the cannula.

21. The self-sealing closure device of claim 20, further comprising an undercut positioned adjacent the tapered surface and partially defining the recess, the undercut extending further in the radial direction than an immediately adjacent portion of the tapered surface, the undercut being positioned and dimensioned to receive a lip coupled to the second end of the cannula.

22. The self-sealing closure device of claim 19, wherein the elongated passageway has a tapered portion positioned adjacent the septum and adapted to selectively receive portions of the septum.

23. The self-sealing closure device of claim 22, wherein the tapered portion is dimensioned to allow the septum to be substantially entirely moved from a position extending into a non-tapered diameter of the passageway.

24. The self-sealing closure device of claim 19, wherein the septum comprises a plurality of elastic flaps.

25. An ophthalmic surgical device comprising:
a tubular sleeve; and
an elastic closure disposed at one end of said sleeve; the elastic enclosure icluding
an elastic passageway having an entry, and
a plurality of cantilevered flaps formed in the passageway below the entry and configured to move between a first position and a second postion to selectively seal the passageway and to meet at a slit.

26. A self-sealing closure device for an ophthalmic cannula, the self-sealing closure device comprising:
an elastic body;
a boss partially defining a recess and having a passageway; and
a septum positioned within the passageway of the boss, portions of the septum extending substantially perpendicular to the wall of the passage way and configured to move within the passageway to selectively seal the passageway.

27. A device as claimed in claim 26, wherein the passageway includes a tapered portion positioned adjacent the septum and adapted to receive the portions of the septum when they are in the second position.

28. An ophthalmic infusion cannula comprising:
a cannula assembly, the cannula assembly including
an elastic coupling means; and
a tubing assembly, the tubing assembly including
a substantially rigid coupling means;
said rigid coupling means being capable to be pressed into said elastic coupling means.

29. An ophthalmic infusion cannula comprising:
a cannula assembly, the cannula assembly further comprising
an elongated tubular sleeve;
an elastic coupling means disposed at one end of said sleeve; and
a passageway formed in said elastic coupling means;
said passageway being in fluid communication with the lumen of said sleeve; and
a tubing assembly, the tubing assembly further comprising
a flexible tubing, having a first end and a second end;
a fluid connector disposed at said first end of said tubing and operable to connect the lumen of said tubing in fluid communication with other surgical apparatus;
a substantially rigid coupling means disposed at said second end of said tubing; and
a passageway formed in said rigid coupling means;
said passageway being in fluid communication with the lumen of said flexible tubing;
said rigid coupling means being capable to be pressed into said elastic coupling means such that said passageway formed in said rigid coupling means is in fluid communication with said passageway formed in said flexible coupling means.

30. An ophthalmic fiberoptic illuminator comprising:
a cannula assembly, the cannula assembly including
an elastic coupling means; and
an optical fiber assembly, the optical fiber assembly including
a substantially rigid coupling means;
said rigid coupling means being capable to be pressed into said elastic coupling means.

31. An ophthalmic fiberoptic illuminator comprising:
a cannula assembly, the cannula assembly further comprising
an elongated tubular sleeve;
an elastic coupling means disposed at one end of said sleeve; and
a passageway formed in said elastic coupling means;
said passageway being aligned with the lumen of said sleeve; and
an optical fiber assembly, the optical fiber assembly further comprising an optical fiber, having a first end and a second end;
a connector means disposed at said first end of said optical fiber and operable to connect said optical fiber to a source of light; and
a substantially rigid coupling means having a passageway therethrough capable to pass said optical fiber;
said second end of said optical fiber being passed through said passageway and projecting a predetermined distance beyond said rigid coupling means; and
said optical fiber being secured to said rigid coupling means in said predetermined position by adhesive or other means;
said rigid coupling means being capable to be pressed into said elastic coupling means such that said second end of said optical fiber passes through the lumen of said tubular sleeve.
